(19) United States
(12) Patent Application Publication
(10) Pub. No.: US 2025/0371710 A1
(43) Pub. Date: Dec. 4, 2025

(54) MULTI-FRAME CONSISTENCY FOR AUTOMATED MAGNETIC RESONANCE IMAGING PRESCRIPTION

(71) Applicant: GE Precision Healthcare LLC, Waukesha, WI (US)

(72) Inventors: Gaspar Delso, Esplugues de Llobregat (ES); Martin Andreas Janich, Munich (DE)

(21) Appl. No.: 18/677,028

(22) Filed: May 29, 2024

(51) Int. Cl. G06T 7/00; A61B 5/00; A61B 5/055; G06V 10/40; G06V 20/50

(52) U.S. Cl. CPC G06T 7/0014; A61B 5/055; A61B 5/7485; G06V 10/40; G06V 20/50; G06T 2207/10016; G06T 2207/10081; G06T 2207/10088; G06T 2207/10116; G06T 2207/10132; G06T 2207/20212; G06T 2207/30048; G06T 2207/30101; G06V 2201/031

(57) ABSTRACT

A computer-implemented method for performing a scan of a subject utilizing a medical imaging system includes acquiring, via a processing system including one or more processors, a series of images of a region of interest of a subject utilizing the medical imaging system. The computer-implemented method also includes automatically identifying, via the processing system, one or more anatomical features in the region of interest in the series of images. The computer-implemented method further includes automatically refining, via the processing system, the one or more anatomical features for use in generating a prescription for a subsequent scan of the region of interest with the medical imaging system. The refining of the one or more anatomical features is based on a consistency in position for the one or more anatomical features identified.

### BACKGROUND

The subject matter disclosed herein relates to medical imaging and, more particularly, to utilizing multi-frame consistency for automated magnetic resonance imaging (MRI) prescription.

Non-invasive imaging technologies allow images of the internal structures or features of a patient/object to be obtained without performing an invasive procedure on the patient/object. In particular, such non-invasive imaging technologies rely on various physical principles (such as the differential transmission of X-rays through a target volume, the reflection of acoustic waves within the volume, the paramagnetic properties of different tissues and materials within the volume, the breakdown of targeted radionuclides within the body, and so forth) to acquire data and to construct images or otherwise represent the observed internal features of the patient/object.

During MRI, when a substance such as human tissue is subjected to a uniform magnetic field (polarizing field B₀), the individual magnetic moments of the spins in the tissue attempt to align with this polarizing field, but precess about it in random order at their characteristic Larmor frequency. If the substance, or tissue, is subjected to a magnetic field (excitation field B₁) which is in the x-y plane and which is near the Larmor frequency, the net aligned moment, or “longitudinal magnetization”, M_{z}, may be rotated, or “tipped”, into the x-y plane to produce a net transverse magnetic moment, Mₜ. A signal is emitted by the excited spins after the excitation signal B₁ is terminated and this signal may be received and processed to form an image.

When utilizing these signals to produce images, magnetic field gradients (Gₓ, G_{y}, and G_{z}) are employed. Typically, the region to be imaged is scanned by a sequence of measurement cycles in which these gradient fields vary according to the particular localization method being used. The resulting set of received nuclear magnetic resonance (NMR) signals are digitized and processed to reconstruct the image using one of many well-known reconstruction techniques.

There is an increasing demand for MRI simplification. This includes the automation of MRI acquisition prescription and analysis. Identification of anatomical landmarks in the images is often a required step for automation. Current techniques rely on identifying desired anatomical features on a static series or on a single temporal frame from a dynamic series. Errors in this step can translate into noticeable system underperformance.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a computer-implemented method for performing a scan of a subject utilizing a medical imaging system is provided. The computer-implemented method includes acquiring, via a processing system including one or more processors, a series of images of a region of interest of a subject utilizing the medical imaging system. The computer-implemented method also includes automatically identifying, via the processing system, one or more anatomical features in the region of interest in the series of images. The computer-implemented method further includes automatically refining, via the processing system, the one or more anatomical features for use in generating a prescription for a subsequent scan of the region of interest with the medical imaging system. The refining of the one or more anatomical features is based on a consistency in position for the one or more anatomical features identified.

In another embodiment, a system for performing a scan of a subject utilizing a MRI system is provided. The system includes a memory encoding processor-executable routines. The system also includes a processing system including one or more processors and configured to access the memory and to execute the processor-executable routines, wherein the processor-executable routines, when executed by the processing system, cause the processing system to perform actions. The actions include acquiring a dynamic series of MRI images of a region of interest of a subject utilizing an MR scanner. The actions also include automatically identifying one or more anatomical features in the region of interest in the dynamic series of MRI images. The actions further include automatically refining the one or more anatomical features for use in generating a prescription for a subsequent scan of the region of interest with the MR scanner, wherein the refining of the one or more anatomical features is based on a consistency in position over time for the one or more anatomical features identified.

In a further embodiment, a non-transitory computer-readable medium, the computer-readable medium including processor-executable code that when executed by a processing system including one or more processors, causes the processing system to perform actions. The actions include acquiring a dynamic series of magnetic resonance imaging (MRI) images of a region of interest of a subject utilizing an MR scanner. The actions also include automatically identifying one or more anatomical features in the region of interest in the dynamic series of MRI images. The actions further include automatically refining the one or more anatomical features for use in generating a prescription for a subsequent scan of the region of interest with the MR scanner, wherein the refining of the one or more anatomical features is based on a consistency in position over time for the one or more anatomical features identified.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 illustrates an embodiment of a magnetic resonance imaging (MRI) system suitable for use with the disclosed technique;

FIG. 2 illustrates a flow diagram of a method for performing a scan of a patient utilizing the MRI system in FIG. 1, in accordance with aspects of the present disclosure;

FIG. 3 illustrates a flow diagram of a method for performing a scan of a patient utilizing the MRI system in FIG. 1 (e.g., utilizing determination of consistency), in accordance with aspects of the present disclosure;

FIG. 4 illustrates a flow diagram of a method for analyzing acquired MR data, in accordance with aspects of the present disclosure;

FIG. 5 illustrates a flow diagram of a method for analyzing acquired MR data (e.g., utilizing fitting function), in accordance with aspects of the present disclosure;

FIG. 6 illustrates a flow diagram of a method for generating an image for prescription generation, in accordance with aspects of the present disclosure;

FIG. 7 illustrates a flow diagram of a method for generating an image for prescription generation (e.g., utilizing weights), in accordance with aspects of the present disclosure;

FIG. 8 illustrates a flow diagram of a method for performing a scan of a patient utilizing the MRI system in FIG. 1 (e.g., utilizing determination of temporal consistency), in accordance with aspects of the present disclosure;

FIG. 9 depicts an MR image of a heart and respective trajectories over time of cardiac valve locations;

FIG. 10 depicts an MR image of a heart and respective trajectories over time utilizing temporal consistency to correct for outliers, in accordance with aspects of the present disclosure; and

FIG. 11 depicts a dynamic series of MR images and the tracking of trajectories over time, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

While aspects of the following discussion are provided in the context of medical imaging, it should be appreciated that the disclosed techniques are not limited to such medical contexts. Indeed, the provision of examples and explanations in such a medical context is only to facilitate explanation by providing instances of real-world implementations and applications. However, the disclosed techniques may also be utilized in other contexts, such as image reconstruction for non-destructive inspection of manufactured parts or goods (i.e., quality control or quality review applications), and/or the non-invasive inspection of packages, boxes, luggage, and so forth (i.e., security or screening applications). In general, the disclosed techniques may be useful in any imaging or screening context or image processing or photography field where a set or type of acquired data undergoes a reconstruction process to generate an image or volume.

Deep-learning (DL) approaches discussed herein may be based on artificial neural networks, and may therefore encompass one or more of deep neural networks, fully connected networks, convolutional neural networks (CNNs), unrolled neural networks, perceptrons, encoders-decoders, recurrent networks, wavelet filter banks, u-nets, general adversarial networks (GANs), dense neural networks, or other neural network architectures. The neural networks may include shortcuts, activations, batch-normalization layers, and/or other features. These techniques are referred to herein as DL techniques, though this terminology may also be used specifically in reference to the use of deep neural networks, which is a neural network having a plurality of layers.

As discussed herein, DL techniques (which may also be known as deep machine learning, hierarchical learning, or deep structured learning) are a branch of machine learning techniques that employ mathematical representations of data and artificial neural networks for learning and processing such representations. By way of example, DL approaches may be characterized by their use of one or more algorithms to extract or model high level abstractions of a type of data-of-interest. This may be accomplished using one or more processing layers, with each layer typically corresponding to a different level of abstraction and, therefore potentially employing or utilizing different aspects of the initial data or outputs of a preceding layer (i.e., a hierarchy or cascade of layers) as the target of the processes or algorithms of a given layer. In an image processing or reconstruction context, this may be characterized as different layers corresponding to the different feature levels or resolution in the data. In general, the processing from one representation space to the next-level representation space can be considered as one ‘stage’ of the process. Each stage of the process can be performed by separate neural networks or by different parts of one larger neural network.

In the following disclosure, the techniques are discussed utilizing MRI data as the example image data. The techniques may also be utilized with other type of imaging data. For example, the input images may be derived from other medical imaging systems (e.g., ultrasound imaging, computed tomography imaging, an X-ray imaging system, etc.).

The present disclosure provides systems and methods for utilizing multi-frame consistency for automated magnetic resonance imaging (MRI) prescription. In particular, a redundancy in position (e.g., multi-frame consistency) in a series (e.g., scanned with one pulse sequence and sharing the same Series Instance UID DICOM attribute) of MR images may be utilized to increase the robustness of anatomical landmark identification and, by extension, of automated prescription. In certain embodiments, the temporal redundancy (e.g., position over time or temporal consistency) of a dynamic MRI series is utilized to increase the robustness of anatomical landmark identification and, by extension, of automated prescription. Additionally, multi-frame consistency and/or temporal consistency can be used as a confidence metric by the automated workflow (e.g., to determine when data reacquisition is required) to provide information as to quality of the acquired images. The disclosed embodiments increase auto-prescription robustness to outliers and correspondingly increase operation confidence. The disclosed embodiments also provide automated notification of low quality acquisitions requiring rescan.

In certain embodiments, anatomical features are extracted independently from several frame images and then combined (e.g., with a median operation). In certain embodiments, these frame images may be weighted (e.g., based the degree of change in position of one or more anatomical landmarks or features (e.g., cardiac valves) of a region of interest (e.g., of heart)). In certain embodiments, the temporal dimension may be utilized in the feature extraction procedure. As noted above, the consistency between the features (e.g. obtained over time) can be leveraged as confidence metric. In certain embodiments, a known function may be fitted to the landmark series, and a fitting error threshold is defined to inform the operator of a need for rescan.

In the disclosed embodiments, a method and system for performing a scan of a subject utilizing a medical imaging system (e.g., magnetic resonance imaging (MRI) system) is provided. The method and system includes acquiring, via a processing system including one or more processors, a series of images (e.g., MRI images) of a region of interest of a subject utilizing the medical imaging system (e.g., an MR scanner). The method and system also includes automatically identifying, via the processing system, one or more anatomical features in the region of interest in the series of images. The method and system further includes automatically refining, via the processing system, the one or more anatomical features (e.g., via determining one or more images from the series of images or generating a combined image from the series of images) for use in generating a prescription for a subsequent scan of the region of interest with the medical imaging system. The determination of the one or more images is based on a consistency in position (e.g., multi-frame consistency) for the one or more anatomical features identified. In certain embodiments, the series of images includes a dynamic series of images (e.g., dynamic series of MR images), and the determination or generation of the one or more images is based on the consistency in position over time (e.g., temporal consistency) for the one or more anatomical features identified.

In certain embodiments, the method and system further include generating, via the processing system, the prescription for the subsequent scan of the region of interest. For example, in certain embodiments, intelligent prescription (e.g., cardiac intelligent prescription) utilizes deep learning algorithms to automatically identify anatomical structures and to prescribe slices for a diagnostic scan based on the one or more images generated based on the consistency in position for the one or more anatomical features identified. An example of intelligent prescription may be AIRx™ from GE HealthCare (e.g., a version specific for the heart).

In certain embodiments, the method and system further include automatically extracting, via the processing system, corresponding anatomical features of the one or more anatomical features identified from each image of the series of images. In certain embodiments, the method and system also include automatically combining, via the processing system, the corresponding anatomical feature of the one or more anatomical features extracted from the series of images using a median operation to generate a combined image for use in generating the prescription. In certain embodiments, each corresponding anatomical feature of the corresponding anatomical features is weighted in the generation of the combined image.

In certain embodiments, the method and system further include automatically determining, via the processing system, the consistency in position for the one or more anatomical features identified in the series of images. In certain embodiments, automatically determining the consistency in position for the one or more anatomical features includes automatically comparing, via the processing system, the one or more anatomical features identified in one image of the series of images to corresponding anatomical features identified in one or more other images of the series of images to determine any respective change in position of the one or more anatomical features identified in the one image. In certain embodiments, the method and system further include calculating, via the processing system, a confidence metric for utilization of the one or more images for prescription generation based on the consistency in position of the one or more anatomical features identified in the series of images.

In certain embodiments, the series of images includes a dynamic series of images, and the one or more other images includes a first image acquired immediately prior to the one image or a second image acquired immediately subsequent to the one image. In certain embodiments, the one image is compared to both the first image and the second image to determine any respective change in position of the one or more anatomical features identified in the one image.

In certain embodiments, the method and system further include identifying, via the processing system, any image of the series of images that lacks a desired consistency in position for the one or more anatomical features identified relative to the other images of the series of images. In certain embodiments, identifying any image of the series of images that lacks the desired consistency in position includes comparing any respective change in position of the one or more anatomical features identified to a model of an expected change for the one or more anatomical features identified. In certain embodiments, the method and system further include correcting, via the processing system, the series of images based on any images identified as lacking the desired consistency in position. In certain embodiments, correcting the series of images includes removing any images identified as lacking the desired consistency in position from the series of images prior to determining the one or more images for use in generating the prescription.

In certain embodiments, the method and system further include applying, via the processing system, a fitting function to the one or more anatomical features identified in the series of images. In certain embodiments, the method and system also include determining, via the processing system, a fitting error based on application of the fitting function to the one or more anatomical features identified in the series of images. In certain embodiments, the method and system also include comparing, via the processing system, the fitting error to a predetermined fitting error threshold. In certain embodiments, the method and system also include when the fitting error meets or exceeds the predetermined fitting error threshold, providing, via the processing system, a user-perceptible notification to a user to perform a rescan to acquire another series of images for potential use in generating the one or more images for use in generating the prescription.

In the disclosed embodiments, a method and system for performing a scan of a subject utilizing a magnetic resonance imaging (MRI) system is provided. The method and system includes acquiring a dynamic series of MRI images of a region of interest of a subject utilizing an MR scanner. The actions also include automatically identifying one or more anatomical features in the region of interest in the dynamic series of MRI images. The actions further include automatically determining one or more images from the dynamic series of MRI images for use in generating a prescription for a subsequent scan of the region of interest with the MR scanner, wherein the determination of the one or more images is based on a consistency in position over time for the one or more anatomical features identified.

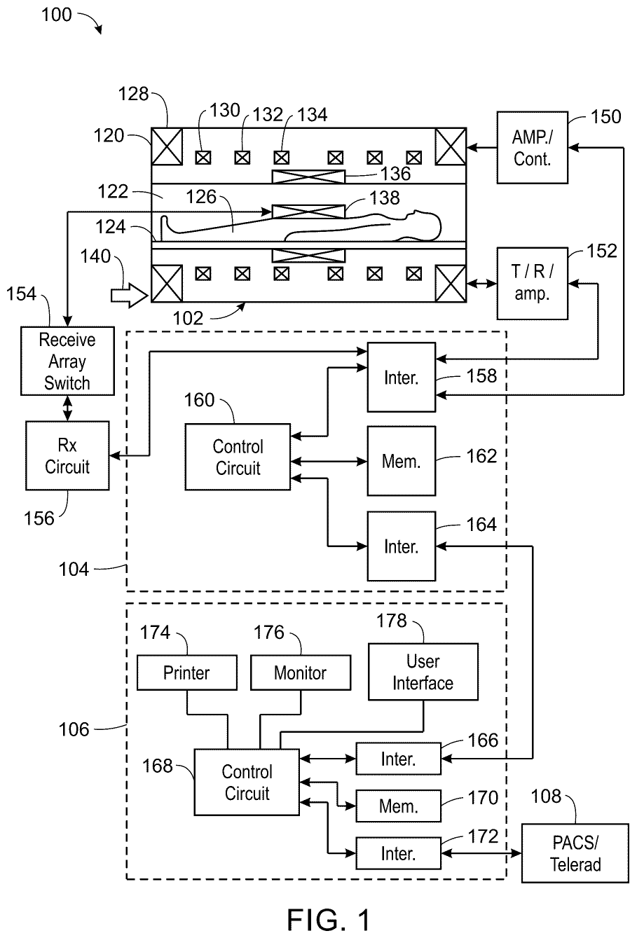

With the preceding in mind, FIG. 1 a magnetic resonance imaging (MRI) system 100 is illustrated schematically as including a scanner 102, scanner control circuitry 104, and system control circuitry 106. According to the embodiments described herein, the MRI system 100 is generally configured to perform MR imaging.

System 100 additionally includes remote access and storage systems or devices such as picture archiving and communication systems (PACS) 108, or other devices such as teleradiology equipment so that data acquired by the system 100 may be accessed on- or off-site. In this way, MR data may be acquired, followed by on- or off-site processing and evaluation. While the MRI system 100 may include any suitable scanner or detector, in the illustrated embodiment, the system 100 includes a full body scanner 102 having a housing 120 through which a bore 122 is formed. A table 124 is moveable into the bore 122 to permit a patient 126 (e.g., subject) to be positioned therein for imaging selected anatomy within the patient.

Scanner 102 includes a series of associated coils for producing controlled magnetic fields for exciting the gyromagnetic material within the anatomy of the patient being imaged. Specifically, a primary magnet coil 128 is provided for generating a primary magnetic field, B₀, which is generally aligned with the bore 122. A series of gradient coils 130, 132, and 134 permit controlled magnetic gradient fields to be generated for positional encoding of certain gyromagnetic nuclei within the patient 126 during examination sequences. A radio frequency (RF) coil 136 (e.g., RF transmit coil) is configured to generate radio frequency pulses for exciting the certain gyromagnetic nuclei within the patient. In addition to the coils that may be local to the scanner 102, the system 100 also includes a set of receiving coils or RF receiving coils 138 (e.g., an array of coils) configured for placement proximal (e.g., against) to the patient 126. As an example, the receiving coils 138 can include cervical/thoracic/lumbar (CTL) coils, head coils, single-sided spine coils, and so forth. Generally, the receiving coils 138 are placed close to or on top of the patient 126 so as to receive the weak RF signals (weak relative to the transmitted pulses generated by the scanner coils) that are generated by certain gyromagnetic nuclei within the patient 126 as they return to their relaxed state.

The various coils of system 100 are controlled by external circuitry to generate the desired field and pulses, and to read emissions from the gyromagnetic material in a controlled manner. In the illustrated embodiment, a main power supply 140 provides power to the primary field coil 128 to generate the primary magnetic field, B₀. A power input (e.g., power from a utility or grid), a power distribution unit (PDU), a power supply (PS), and a driver circuit 150 may together provide power to pulse the gradient field coils 130, 132, and 134. The driver circuit 150 may include amplification and control circuitry for supplying current to the coils as defined by digitized pulse sequences output by the scanner control circuitry 104.

Another control circuit 152 is provided for regulating operation of the RF coil 136. Circuit 152 includes a switching device for alternating between the active and inactive modes of operation, wherein the RF coil 136 transmits and does not transmit signals, respectively. Circuit 152 also includes amplification circuitry configured to generate the RF pulses. Similarly, the receiving coils 138 are connected to switch 154, which is capable of switching the receiving coils 138 between receiving and non-receiving modes. Thus, the receiving coils 138 resonate with the RF signals produced by relaxing gyromagnetic nuclei from within the patient 126 while in the receiving mode, and they do not resonate with RF energy from the transmitting coils (i.e., coil 136) so as to prevent undesirable operation while in the non-receiving mode. Additionally, a receiving circuit 156 is configured to receive the data detected by the receiving coils 138 and may include one or more multiplexing and/or amplification circuits.

It should be noted that while the scanner 102 and the control/amplification circuitry described above are illustrated as being coupled by a single line, many such lines may be present in an actual instantiation. For example, separate lines may be used for control, data communication, power transmission, and so on. Further, suitable hardware may be disposed along each type of line for the proper handling of the data and current/voltage. Indeed, various filters, digitizers, and processors may be disposed between the scanner and either or both of the scanner and system control circuitry 104, 106.

As illustrated, scanner control circuitry 104 includes an interface circuit 158, which outputs signals for driving the gradient field coils and the RF coil and for receiving the data representative of the magnetic resonance signals produced in examination sequences. The interface circuit 158 is coupled to a control and analysis circuit 160. The control and analysis circuit 160 executes the commands for driving the circuit 150 and circuit 152 based on defined protocols selected via system control circuit 106.

Control and analysis circuit 160 also serves to receive the magnetic resonance signals and performs subsequent processing before transmitting the data to system control circuit 106. Scanner control circuit 104 also includes one or more memory circuits 162, which store configuration parameters, pulse sequence descriptions, examination results, and so forth, during operation.

Interface circuit 164 is coupled to the control and analysis circuit 160 for exchanging data between scanner control circuitry 104 and system control circuitry 106. In certain embodiments, the control and analysis circuit 160, while illustrated as a single unit, may include one or more hardware devices. The system control circuit 106 includes an interface circuit 166, which receives data from the scanner control circuitry 104 and transmits data and commands back to the scanner control circuitry 104. The control and analysis circuit 168 may include a CPU in a multi-purpose or application specific computer or workstation. Control and analysis circuit 168 is coupled to a memory circuit 170 to store programming code for operation of the MRI system 100 and to store the processed image data for later reconstruction, display and transmission. The programming code may execute one or more algorithms that, when executed by a processor, are configured to perform reconstruction of acquired data as described below. In certain embodiments, the memory circuit 170 may store one or more neural networks for prescription of a scan as described below and/or image reconstruction. In certain embodiments, the memory circuit 170 may stores one or more algorithms for generating one or more images from a series of MRI images for use in generating a prescription for a scan based on a consistency (e.g., multi-frame consistency and/or temporal consistency) in position for one or more identified features as described in the techniques herein. In certain embodiments, image reconstruction may occur on a separate computing device having processing circuitry and memory circuitry.

A processing component (e.g., a microprocessor or processing circuitry) and a memory of the magnetic resonance imaging system 100, such as may be present in scanner control circuitry 104 and/or system control circuitry 106, may be used to execute stored software code, instructions, or routines for acquiring and processing the MR data. The term “code” or “software code” used herein refers to any instructions or set of instructions that control the magnetic resonance imaging system 100. The code or software code may exist in a computer-executable form, such as machine code, which is the set of instructions and data directly executed by the processing component of the scanner control circuitry 104 and/or system control circuitry 106, human-understandable form, such as source code, which may be compiled in order to be executed by the processing component of the scanner control circuitry 104 and/or system control circuitry 106, or an intermediate form, such as object code, which is produced by a compiler. In some embodiments, the magnetic resonance imaging system 100 may include a plurality of controllers.

As an example, the memory may store processor-executable software code or instructions (e.g., firmware or software), which are tangibly stored on a non-transitory computer readable medium. Additionally or alternatively, the memory may store data. As an example, the memory may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, processing component may include multiple microprocessors, one or more “general-purpose” microprocessors, one or more special-purpose microprocessors, and/or one or more application specific integrated circuits (ASICS), or some combination thereof. For example, the processing component may include one or more reduced instruction set (RISC) or complex instruction set (CISC) processors. The processing component may include multiple processors, and/or the memory may include multiple memory devices.

In certain embodiments, the processing component may be configured to utilizing multi-frame consistency and/or temporal consistency for automated magnetic resonance imaging (MRI) prescription. In certain embodiments, the processing component may be configured to acquire a series of MRI images of a region of interest of a subject utilizing an MR scanner. The processing component may be configured to automatically identify one or more anatomical features in the region of interest in the series of MRI images. The processing component may be configured to automatically determine one or more images from the series of MRI images for use in generating a prescription for a subsequent scan of the region of interest with the MR scanner. The determination of the one or more images is based on a consistency in position (e.g., multi-frame consistency) for the one or more anatomical features identified. In certain embodiments, the series of MRI images includes a dynamic series of MRI images, and the determination of the one or more images is based on the consistency in position over time (e.g., temporal consistency) for the one or more anatomical features identified.

In certain embodiments, the processing component may be configured to generate the prescription for the subsequent scan of the region of interest. In certain embodiments, the processing component may be configured to automatically extract corresponding anatomical features of the one or more anatomical features identified from each MRI image of the series of MRI images. In certain embodiments, the processing component may be configured to automatically combine the corresponding anatomical feature of the one or more anatomical features extracted from the series of MRI images using a median operation to generate a combined image for use in generating the prescription. In certain embodiments, each corresponding anatomical feature of the corresponding anatomical features is weighted in the generation of the combined image.

In certain embodiments, the processing component may be configured to automatically determine the consistency in position for the one or more anatomical features identified in the series of MRI images. In certain embodiments, automatically determining the consistency in position for the one or more anatomical features includes automatically comparing the one or more anatomical features identified in one MRI image of the series of MRI images to corresponding anatomical features identified in one or more other MRI images of the series of MRI images to determine any respective change in position of the one or more anatomical features identified in the one MRI image. In certain embodiments, the processing component may be configured to calculate a confidence metric for utilization of the one or more images for prescription generation based on the consistency in position of the one or more anatomical features identified in the series of MRI images.

In certain embodiments, the series of MRI images includes a dynamic series of MRI images, and the one or more other MRI images includes a first MRI image acquired immediately prior to the one MRI image or a second MRI image acquired immediately subsequent to the one MRI image. In certain embodiments, the one MRI image is compared to both the first MRI image and the second MRI image to determine any respective change in position of the one or more anatomical features identified in the one MRI image.

In certain embodiments, the processing component may be configured to identify any MRI image of the series of MRI images that lacks a desired consistency in position for the one or more anatomical features identified relative to the other MRI images of the series of MRI images. In certain embodiments, identifying any MRI image of the series of MRI images that lacks the desired consistency in position includes comparing any respective change in position of the one or more anatomical features identified to a model of an expected change for the one or more anatomical features identified. In certain embodiments, the processing component may be configured to correct the series of MRI images based on any MRI images identified as lacking the desired consistency in position. In certain embodiments, correcting the series of MRI images includes removing any MRI images identified as lacking the desired consistency in position from the series of MRI images prior to determining the one or more images for use in generating the prescription.

In certain embodiments, the processing component may be configured to apply a fitting function to the one or more anatomical features identified in the series of MRI images. In certain embodiments, the processing component may be configured to determine a fitting error based on application of the fitting function to the one or more anatomical features identified in the series of MRI images. In certain embodiments, the processing component may be configured to also include compare the fitting error to a predetermined fitting error threshold. In certain embodiments, the processing component may be configured, when the fitting error meets or exceeds the predetermined fitting error threshold, to provide a user-perceptible notification to a user to perform a rescan to acquire another series of MRI images for potential use in generating the one or more images for use in generating the prescription.

In the disclosed embodiments, the processing component may be configured to acquire a dynamic series of MRI images of a region of interest of a subject utilizing an MR scanner. The processing component may be configured to automatically identify one or more anatomical features in the region of interest in the dynamic series of MRI images. The processing component may be configured to automatically determine one or more images from the dynamic series of MRI images for use in generating a prescription for a subsequent scan of the region of interest with the MR scanner, wherein the determination of the one or more images is based on a consistency in position over time for the one or more anatomical features identified.

An additional interface circuit 172 may be provided for exchanging image data, configuration parameters, and so forth with external system components such as remote access and storage devices 108. Finally, the system control and analysis circuit 168 may be communicatively coupled to various peripheral devices for facilitating operator interface and for producing hard copies of the reconstructed images. In the illustrated embodiment, these peripherals include a printer 174, a monitor 176, and user interface 178 including devices such as a keyboard, a mouse, a touchscreen (e.g., integrated with the monitor 176), and so forth.

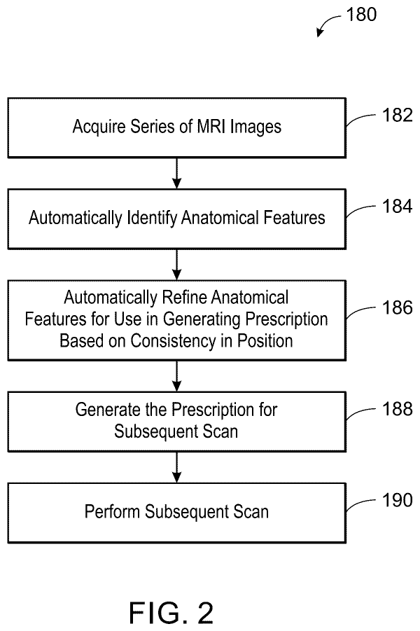

FIG. 2 illustrates a flow diagram of a method 180 for performing a scan of a patient utilizing the MRI system 100 in FIG. 1. One or more steps of the method 180 may be performed by processing circuitry of the magnetic resonance imaging system 100 in FIG. 1. One or more of the steps of the method 180 may be performed simultaneously or in a different order from the order depicted in FIG. 2.

The method 180 includes acquiring a series of MRI images of a region of interest of a subject utilizing an MR scanner (block 182). In certain embodiments, the series of MRI images may be a static series of MRI images. In certain embodiments, the series of MRI images may be a dynamic series of MRI images (or frame images) (e.g., from a cine acquisition). In certain embodiments, the dynamic series of MRI images maybe short axis images. In certain embodiments, the region of interest may be heart. In certain embodiments, the region of interest may be a region different from the heart.

The method 180 also includes automatically identifying one or more anatomical landmarks or features in the region of interest in the series of MRI images (block 184). In certain embodiments, when the region of interest is the heart, the one or more anatomical landmarks or features may be cardiac valves (e.g., tricuspid valve, pulmonary valve, mitral valve, and/or aortic valve). The method 180 further includes automatically refining the one or more anatomical features (e.g., generating one or more images from the series of MRI images) for use in generating a prescription for a subsequent scan of the region of interest with the MR scanner (block 186). The refining of the one or more anatomical features (e.g., generation of the one or more images) is based on a consistency in position (e.g., multi-frame consistency) for the one or more anatomical features identified. In certain embodiments, when the series of MRI images is a dynamic series of MRI images, the generation of the one or more images is based on the consistency in position over time (e.g., temporal consistency) for the one or more anatomical features identified.

In certain embodiments, a combined image (to be utilized for prescription) may be generated from the series of MRI images based on the consistency in position for the one or more anatomical features. For example, the one or more anatomical features may be independently extracted from each image of the series of MRI images and the corresponding anatomical features combined (e.g., utilizing a median operator). In certain embodiments, the corresponding anatomical features may be weighted on the degree of change in position for an anatomical feature in a respective image relative to the other images within the series of MRI images. In particulars, a feature in an image (or frame image) with no change or less of a change in position is given more weight and a feature in an image (or frame image) with greater change in position given less or no weight.

In certain embodiments, the one or more images to be utilized for prescription may be an updated or corrected series from the series of MRI images. In certain embodiments, an outlier (e.g., outlier image) may be identified and corrected, wherein the outlier has too much of a change in position for one or more identified anatomical features. In certain embodiments, an identified outlier may be removed or discarded and not utilized for prescription.

The method 180 includes generating (e.g., automatically) the prescription for the subsequent scan of the region of interest (block 188). The prescription may include prescription parameters and/or a geometry plane (e.g., for slices). For example, in certain embodiments, intelligent prescription (e.g., cardiac intelligent prescription) utilizes deep learning algorithms to automatically identify anatomical structures and to prescribe slices for a diagnostic scan based on the one or more images generated based on the consistency in position for the one or more anatomical features identified. An example of intelligent prescription may be AIRx™ from GE HealthCare (e.g., a version specific for the heart). In certain embodiments, in the case of the heart, a 4-chamber plane intersecting and the mitral and tricuspid valves may be prescribed. The method 180 also includes performing the subsequent scan of the region of interest with the MR scanner utilizing the prescription (block 190).

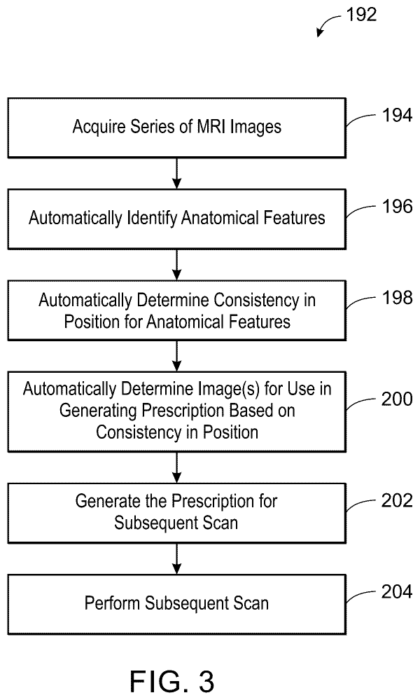

FIG. 3 illustrates a flow diagram of a method 192 for performing a scan of a patient utilizing the MRI system 100 in FIG. 1 (e.g., utilizing determination of consistency). One or more steps of the method 192 may be performed by processing circuitry of the magnetic resonance imaging system 100 in FIG. 1. One or more of the steps of the method 192 may be performed simultaneously or in a different order from the order depicted in FIG. 3.

The method 192 includes acquiring a series of MRI images of a region of interest of a subject utilizing an MR scanner (block 194). In certain embodiments, the series of MRI images may be a static series of MRI images. In certain embodiments, the series of MRI images may be a dynamic series of MRI images (or frame images) (e.g., from a cine acquisition). In certain embodiments, the dynamic series of MRI images maybe short axis images. In certain embodiments, the region of interest may be heart. In certain embodiments, the region of interest may be a region different from the heart.

The method 192 also includes automatically identifying one or more anatomical landmarks or features in the region of interest in the series of MRI images (block 196). In certain embodiments, when the region of interest is the heart, the one or more anatomical landmarks or features may be cardiac valves (e.g., tricuspid valve, pulmonary valve, mitral valve, and/or aortic valve).

The method 192 further includes automatically determining the consistency (e.g., at least multi-frame consistency) in position for the one or more anatomical landmarks or features for the one or more anatomical landmarks or features identified in the series of MRI images (block 198). In certain embodiments, when the series of MRI images is a dynamic series of MRI images, the consistency also includes a consistency in position over time (e.g., temporal consistency). In certain embodiments, determining consistency includes comparing the one or more anatomical features identified in one MRI image of the series of MRI images to corresponding anatomical features identified in one or more MRI images of the series of MRI images to determine any respective change in position of the one or more anatomical features identified in the one MRI image. In certain embodiments, when the series of MRI images is a dynamic series of MRI images, the one or other MRI images may be the MRI image (or frame image) acquired immediately prior to the one MRI image (i.e., the image being analyzed for consistency) and/or the MRI image (or frame image) acquired immediately subsequent to the one MRI image (i.e., the image being analyzed for consistency). In the case where the image to be analyzed for consistency is the first image acquired in the dynamic series of MRI images, it is compared to the image acquired immediately after. In the case where the image to be analyzed for consistency is the last image acquired in the dynamic series of MRI images, it is compared to the image acquired immediately before. In the case where the image to be analyzed is neither the first or last image acquired in the dynamic series of MRI images, it is compared both to the image acquired immediately before and the image acquired immediately after.

The method 192 further includes automatically determining one or more images from the series of MRI images for use in generating a prescription for a subsequent scan of the region of interest with the MR image (block 200). The determination of the one or more images is based on a consistency in position (e.g., multi-frame consistency) for the one or more anatomical features identified. In certain embodiments, when the series of MRI images is a dynamic series of MRI images, the determination of the one or more images is based on the consistency in position over time (e.g., temporal consistency) for the one or more anatomical features identified.

In certain embodiments, a combined image (to be utilized for prescription) may be generated from the series of MRI images based on the consistency in position for the one or more anatomical features. For example, the one or more anatomical features may be independently extracted from each image of the series of MRI images and the corresponding anatomical features combined (e.g., utilizing a median operator). In certain embodiments, the corresponding anatomical features may be weighted on the degree of change in position for an anatomical feature in a respective image relative to the other images within the series of MRI images. In particulars, a feature in an image (or frame image) with no change or less of a change in position is given more weight and a feature in an image (or frame image) with greater change in position given less or no weight.

In certain embodiments, the one or more images to be utilized for prescription may be an updated or corrected series from the series of MRI images. In certain embodiments, an outlier (e.g., outlier image) may be identified and corrected, wherein the outlier has too much of a change in position for one or more identified anatomical features. In certain embodiments, an identified outlier may be removed or discarded and not utilized for prescription.

The method 192 includes generating (e.g., automatically) the prescription for the subsequent scan of the region of interest (block 202). The prescription may include prescription parameters and/or a geometry plane (e.g., for slices). For example, in certain embodiments, intelligent prescription (e.g., cardiac intelligent prescription) utilizes deep learning algorithms to automatically identify anatomical structures and to prescribe slices for a diagnostic scan based on the one or more images generated based on the consistency in position for the one or more anatomical features identified. The method 192 also includes performing the subsequent scan of the region of interest with the MR scanner utilizing the prescription (block 204).

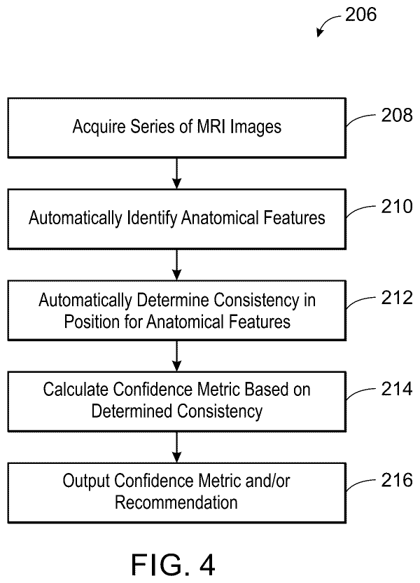

FIG. 4 illustrates a flow diagram of a method 206 for analyzing acquired MR data. One or more steps of the method 206 may be performed by processing circuitry of the magnetic resonance imaging system 100 in FIG. 1. One or more of the steps of the method 206 may be performed simultaneously or in a different order from the order depicted in FIG. 4.

The method 206 includes acquiring a series of MRI images of a region of interest of a subject utilizing an MR scanner (block 208). In certain embodiments, the series of MRI images may be a static series of MRI images. In certain embodiments, the series of MRI images may be a dynamic series of MRI images (or frame images) (e.g., from a cine acquisition). In certain embodiments, the dynamic series of MRI images maybe short axis images. In certain embodiments, the region of interest may be heart. In certain embodiments, the region of interest may be a region different from the heart.

The method 206 also includes automatically identifying one or more anatomical landmarks or features in the region of interest in the series of MRI images (block 210). In certain embodiments, when the region of interest is the heart, the one or more anatomical landmarks or features may be cardiac valves (e.g., tricuspid valve, pulmonary valve, mitral valve, and/or aortic valve).

The method 206 further includes automatically determining the consistency (e.g., at least multi-frame consistency) in position for the one or more anatomical landmarks or features for the one or more anatomical landmarks or features identified in the series of MRI images (block 212). In certain embodiments, when the series of MRI images is a dynamic series of MRI images, the consistency also includes a consistency in position over time (e.g., temporal consistency). Determining the consistency may occur as described in the method 192 in FIG. 3.

The method 206 even further includes calculating a confidence metric for utilization of the one or more images for prescription generation (or another task) based the determined consistency in position for the one or more anatomical features (block 214). The confidence metric is indicative of the quality of the acquired MRI image data and its usefulness for prescription. In certain embodiments, the confidence metric may be a confidence limit, confidence interval, or some other metric. In certain embodiments, the confidence metric may be a numerical value or a number associated with a scale. A higher score may be associated with higher quality data and a lower score associated with lower quality data. In certain embodiments, the confidence metric maybe an expression (e.g., low, medium, or high). The method 206 also includes outputting or providing a user-perceptible the confidence metric and/or a recommendation (block 216). The recommendation may be a suggestion to reacquire another series of MRI images for utilization in generation of the prescription. In certain embodiments, the confidence metric and the recommendation may not be provided when the series of MRI images are substantially consistent (which is indicative of higher image quality).

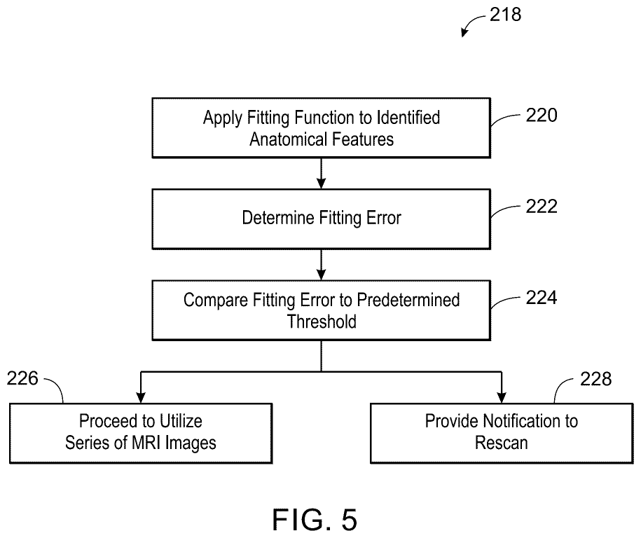

FIG. 5 illustrates a flow diagram of a method 218 for analyzing acquired MR data (e.g., utilizing fitting function). One or more steps of the method 218 may be performed by processing circuitry of the magnetic resonance imaging system 100 in FIG. 1. One or more of the steps of the method 218 may be performed simultaneously or in a different order from the order depicted in FIG. 5.

The method 218 includes applying a fitting function to one or more anatomical features identified in the series of MRI images (block 220). In certain embodiments, the fitting function may be applied to a relative position of the anatomical features. In certain embodiments, the fitting function may be applied to an image quality metric of the anatomical features. The method 218 also includes determining a fitting error based on application of the fitting function to the one or more anatomical features identified in the series of MRI images (block 222). The method 218 further includes comparing the fitting error to a predetermined threshold (e.g., fitting error threshold) (block 224). When the fitting error is less than the predetermined threshold, the method 218 includes proceeding to utilize the series of MRI images (e.g., for generation of prescription or other task) (block 226). When the fitting error meets or exceeds the predetermined fitting error threshold, providing a user-perceptible notification to a user to perform a rescan to acquire another series of MRI images (e.g., for generation of prescription or other task) (block 228).

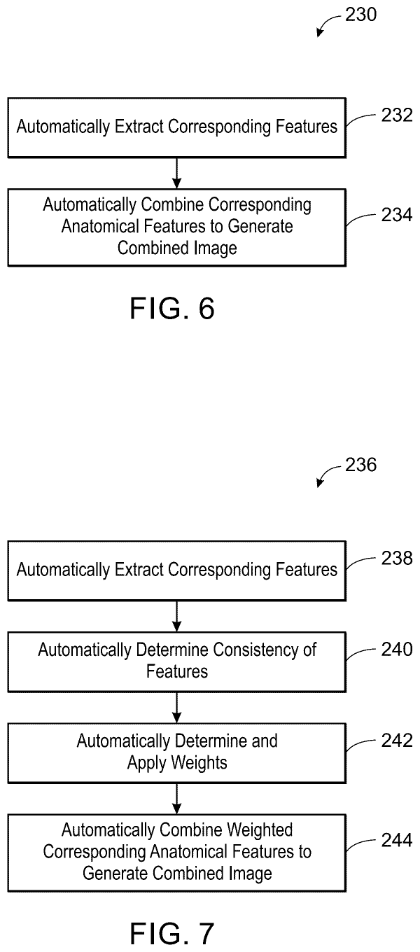

FIG. 6 illustrates a flow diagram of a method 230 for generating an image for prescription generation. One or more steps of the method 230 may be performed by processing circuitry of the magnetic resonance imaging system 100 in FIG. 1. One or more of the steps of the method 230 may be performed simultaneously or in a different order from the order depicted in FIG. 6.

The method 230 includes automatically independently extracting one or more corresponding anatomical features from each image of the series of MRI images (block 232). The method 230 also includes automatically combining (e.g., utilizing a median operation) the corresponding one or more anatomical features to generate a combined image (e.g., for generation of prescription or other task) (block 234). Utilizing the median corrects any undesired inconsistency in position of one or more anatomical features by reducing the impact of the undesired inconsistency.

FIG. 7 illustrates a flow diagram of a method 236 for generating an image for prescription generation (e.g., utilizing weights). One or more steps of the method 236 may be performed by processing circuitry of the magnetic resonance imaging system 100 in FIG. 1. One or more of the steps of the method 236 may be performed simultaneously or in a different order from the order depicted in FIG. 7.

The method 236 includes automatically independently extracting one or more corresponding anatomical features from each image of the series of MRI images (block 238). The method 236 also includes automatically determining a consistency in position for the one or more corresponding anatomical features (e.g., between the images and/or over time (if dynamic series of MRI images)) (block 240). The method 236 includes automatically determining and applying weights to each individual anatomical feature (of a respective image) of the corresponding anatomical features based on the degree of change in position for the anatomical feature in the respective image relative to the other images within the series of MRI images (block 242). In particulars, a feature in an image (or frame image) with no change or less of a change in position is given more weight and a feature in an image (or frame image) with greater change in position given less or no weight.

The method 236 even further includes automatically combining (e.g., utilizing a median operation) the weighted corresponding one or more anatomical features to generate a combined image (e.g., for generation of prescription or other task) (block 244). Utilizing the median corrects any undesired inconsistency in position of one or more anatomical features by reducing the impact of the undesired inconsistency.

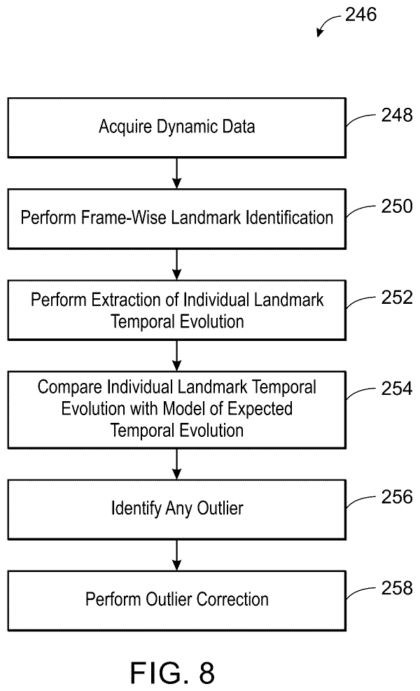

FIG. 8 illustrates a flow diagram of a method 246 for performing a scan of a patient utilizing the MRI system in FIG. 1 (e.g., utilizing determination of temporal consistency). One or more steps of the method 246 may be performed by processing circuitry of the magnetic resonance imaging system 100 in FIG. 1. One or more of the steps of the method 246 may be performed simultaneously or in a different order from the order depicted in FIG. 8.

The method 246 includes acquiring a dynamic series of MRI images (e.g., frame images) of a region of interest of a subject utilizing an MR scanner (block 248). In certain embodiments, the dynamic series of MRI images maybe short axis images. In certain embodiments, the region of interest may be heart. In certain embodiments, the region of interest may be a region different from the heart.

The method 246 also includes automatically performing frame-wise landmark or feature extraction of one or more anatomical landmarks or features in each image of the dynamic series of MRI images (block 250). In certain embodiments, when the region of interest is the heart, the one or more anatomical landmarks or features may be cardiac valves (e.g., tricuspid valve, pulmonary valve, mitral valve, and/or aortic valve).

The method 246 further includes performing extraction of individual landmark temporal evolution for each extracted feature of image (block 252). The extraction may occur by determining the consistency in position over time for each individual landmark by comparing the one or more anatomical features identified in one MRI image of the dynamic series of MRI images to corresponding anatomical features identified in one or more MRI images of the dynamic series of MRI images to determine any respective change in position of the one or more anatomical features identified in the one MRI image. An extracted anatomical features of a respective MRI image of the dynamic series of MRI images is compared to the corresponding extracted anatomical feature of the MRI image (or frame image) acquired immediately prior to the respective MRI image (i.e., the image being analyzed for consistency) and/or the MRI image (or frame image) acquired immediately subsequent to the respective MRI image (i.e., the image being analyzed for consistency). In the case where the extracted feature to be analyzed for consistency is from the first image acquired in the dynamic series of MRI images, it is compared to corresponding extracted feature of the image acquired immediately after. In the case where the extracted feature to be analyzed for consistency is from the last image acquired in the dynamic series of MRI images, it is compared to corresponding extracted feature of the image acquired immediately before. In the case where extracted feature of the image to be analyzed is from neither the first or last image acquired in the dynamic series of MRI images, it is compared both to the corresponding extracted feature from the image acquired immediately before and the image acquired immediately after.

The method 246 even further includes comparing the individual landmark temporal evolution with a model of expected temporal evolution (block 254). This model is utilized to calculate the expected movement of the organ, e.g. the heart, and therefore constraining motion within physiologically meaningful ranges. The method 246 still further includes identifying any outlier for any of the individual landmark temporal evolutions based on the comparison (block 256). In certain embodiments, an image may be identified as an outlier if it deviates from the model by an amount equal to or greater than a predetermined threshold amount. The method 246 further includes performing outlier correction (block 258). In certain embodiments, correction includes removing the image or image frame with the outlier individual landmark temporal evolution from the series of dynamic MRI images. In certain embodiments, the outlier individual landmark temporal evolution may be corrected via a motion correction technique performed on the image with the outlier.

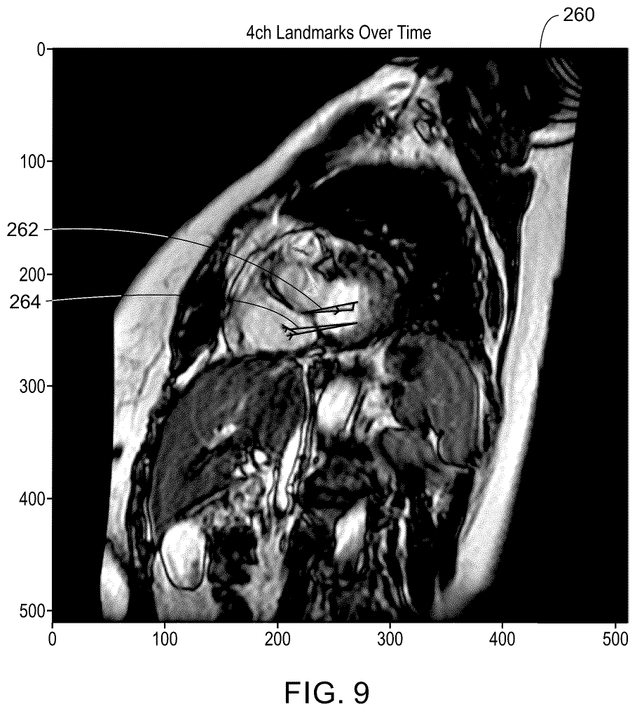

FIG. 9 depicts an MR image 260 of a heart and respective trajectories over time of cardiac valve locations. The MR image 260 is a shot axis image. The MR image 260 of four chambers (4CH). The trajectories were tracked across a dynamic short axis series. Trajectory 262 is of the mitral valve. Trajectory 264 is of the tricuspid valve. Both trajectories 262 and 264 include outliers caused by image quality issues.

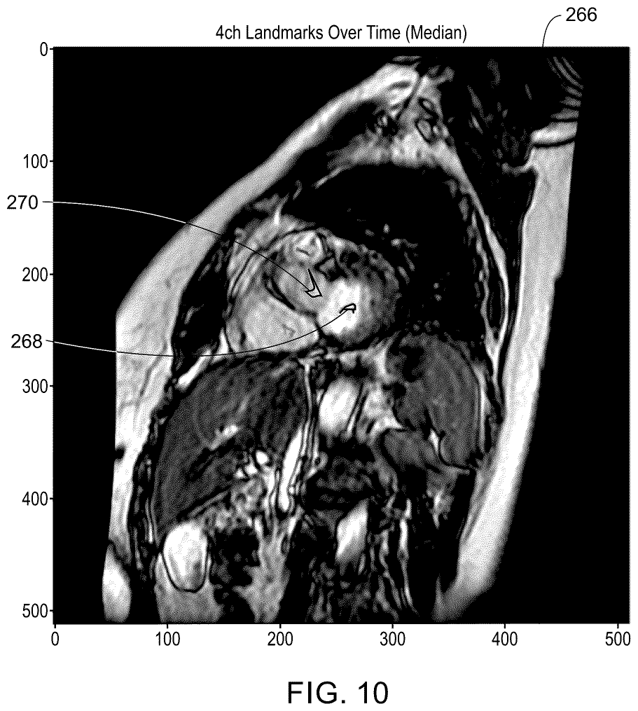

FIG. 10 depicts an MR image 266 of a heart and respective trajectories over time utilizing temporal consistency to correct for outliers. The MR image 266 is median image derived from the same dynamic short axis series in FIG. 9. Any outlier detected utilizing the techniques described above (e.g., the method 246 in FIG. 8) is corrected (e.g., removed). Trajectory 268 is of the mitral valve. Trajectory 270 is of the tricuspid valve. Both trajectories 268 and 270 lack the outliers and, thus, the image quality issues as seen in FIG. 9.

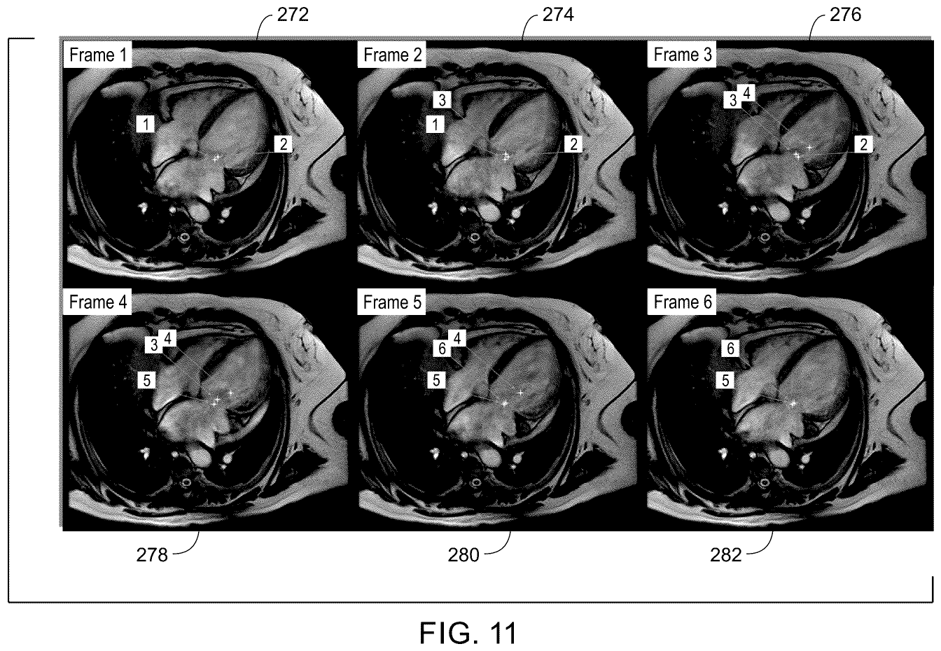

FIG. 11 depicts a dynamic series of MR images 272, 274, 276, 278, 280, and 282 (e.g., frame images) and the tracking of trajectories over time. The MR images 272, 274, 276, 278, 280, and 282 are a temporal series acquired sequentially. MR image 272 is the first frame image. MR image 274 is the second frame image. MR image 276 is the third frame image. MR image 278 is the fourth frame image. MR image 280 is the fifth frame image. MR image 282 is the second frame image. In the temporal series, the mitral valve is the identified anatomical landmark in each MR image that is being compared to temporally adjacent MR images. For example, since the MR image 272 is temporally the first image, the position of the mitral valve (identified as 1) is compared to the position of the mitral valve in MR image 274 (identified as 2). The position of the mitral valve in MR image 274 (identified as 2) is compared to the position of the mitral valve in MR image 272 (identified as 1) and the position of the mitral valve in MR image 276 (identified as 3). The position of the mitral valve in MR image 276 (identified as 3) is compared to the position of the mitral valve in MR image 274 (identified as 2) and the position of the mitral valve in MR image 278 (identified as 4). The position of the mitral valve in MR image 278 (identified as 4) is compared to the position of the mitral valve in MR image 276 (identified as 3) and the position of the mitral valve in MR image 280 (identified as 5). The position of the mitral valve in MR image 280 (identified as 5) is compared to the position of the mitral valve in MR image 278 (identified as 4) and the position of the mitral valve in MR image 282 (identified as 6). Since the MR image 282 is temporally the last image, the position of the mitral valve in MR image 282 (identified as 6) is compared to the position of the mitral valve in MR image 280 (identified as 5).

In certain embodiments, in the disclosed techniques, instead of utilizing changes in position of the identified anatomical landmarks (e.g., over time), images in the series of MRI images may be considered outliers and removed if they have poor image quality relative to the other images in the series. In certain embodiments, the disclosed techniques may be utilized to remove (e.g., comparison of position of anatomical landmarks in heart) may be utilized to automatically detect the systolic and diastolic phases.

Technical effects of the disclosed subject matter include providing systems and methods for utilizing multi-frame consistency for automated magnetic resonance imaging (MRI) prescription. In particular, a redundancy in position (e.g., multi-frame consistency) in a series (e.g., scanned with one pulse sequence and sharing the same Series Instance UID DICOM attribute) of MR images may be utilized to increase the robustness of anatomical landmark identification and, by extension, of automated prescription. Technical effects of the disclosed embodiments include utilizing the temporal redundancy (e.g., position over time or temporal consistency) of a dynamic MRI series to increase the robustness of anatomical landmark identification and, by extension, of automated prescription. Technical effects of the disclosed embodiments include utilizing multi-frame consistency and/or temporal consistency as a confidence metric by the automated workflow (e.g., to determine when data reacquisition is required) to provide information as to quality of the acquired images. Technical effects of the disclosed embodiments include increasing auto-prescription robustness to outliers and correspondingly increasing operation confidence.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A computer-implemented method for performing a scan of a subject utilizing a medical imaging system, comprising:
acquiring, via a processing system comprising one or more processors, a series of images of a region of interest of a subject utilizing the medical imaging system;
automatically identifying, via the processing system, one or more anatomical features in the region of interest in the series of images; and
automatically refining, via the processing system, the one or more anatomical features for use in generating a prescription for a subsequent scan of the region of interest with the medical imaging system, wherein the refining of the one or more anatomical features is based on a consistency in position for the one or more anatomical features identified.

2. The computer-implemented method of claim 1, wherein the medical imaging comprises a magnetic resonance imaging system.

3. The computer-implemented method of claim 1, wherein the medical imaging system comprises an ultrasound system.

4. The computer-implemented method of claim 1, wherein the medical imaging system comprises an X-ray system.

5. The computer-implemented method of claim 1, wherein the medical imaging system comprises a computed tomography imaging system.

6. The computer-implemented method of claim 1, further comprising generating, via the processing system, the prescription for the subsequent scan of the region of interest.

7. The computer-implemented method of claim 1, further comprising:
automatically extracting, via the processing system, corresponding anatomical features of the one or more anatomical features identified from each image of the series of images; and
automatically combining, via the processing system, the corresponding anatomical feature of the one or more anatomical features extracted from the series of images using a median operation to generate a combined image for use in generating the prescription.

8. The computer-implemented method of claim 7, wherein each corresponding anatomical feature of the corresponding anatomical features is weighted in the generation of the combined image.

9. The computer-implemented method of claim 1, further comprising automatically determining, via the processing system, the consistency in position for the one or more anatomical features identified in the series of images by automatically comparing, via the processing system, the one or more anatomical features identified in one image of the series of images to corresponding anatomical features identified in one or more other images of the series of images to determine any respective change in position of the one or more anatomical features identified in the one image.

10. The computer-implemented method of claim 9, wherein the series of images comprises a dynamic series of images, and the one or more other images comprises a first image acquired immediately prior to the one image or a second image acquired immediately subsequent to the one image.

11. The computer-implemented method of claim 10, wherein the one image is compared to both the first image and the second image to determine any respective change in position of the one or more anatomical features identified in the one image.

12. The computer-implemented method of claim 9, further comprising identifying, via the processing system, any image of the series of images that lacks a desired consistency in position for the one or more anatomical features identified relative to the other images of the series of images.

13. The computer-implemented method of claim 12, wherein identifying any image of the series of images that lacks the desired consistency in position comprises comparing any respective change in position of the one or more anatomical features identified to a model of an expected change for the one or more anatomical features identified.

14. The computer-implemented method of claim 12, further comprising correcting, via the processing system, the series of images based on any images identified as lacking the desired consistency in position.

15. The computer-implemented method of claim 12, wherein correcting the series of images comprises removing any images identified as lacking the desired consistency in position from the series of images prior to determining the one or more images for use in generating the prescription.

16. The computer-implemented method of claim 9, further comprising calculating, via the processing system, a confidence metric for utilization of the one or more images for prescription generation based on the consistency in position of the one or more anatomical features identified in the series of images.

17. The computer-implemented method of claim 1, further comprising:
applying, via the processing system, a fitting function to the one or more anatomical features identified in the series of images;
determining, via the processing system, a fitting error based on application of the fitting function to the one or more anatomical features identified in the series of images;
comparing, via the processing system, the fitting error to a predetermined fitting error threshold; and
when the fitting error meets or exceeds the predetermined fitting error threshold, providing, via the processing system, a user-perceptible notification to a user to perform a rescan to acquire another series of images for potential use in generating the one or more images for use in generating the prescription.

18. The computer-implemented method of claim 1, wherein the series of images comprises a dynamic series of images, and the determination or generation of the one or more images is based on the consistency in position over time for the one or more anatomical features identified.

19. A system for performing a scan of a subject utilizing a magnetic resonance imaging (MRI) system, comprising:
a memory encoding processor-executable routines; and
a processing system comprising one or more processors and configured to access the memory and to execute the processor-executable routines, wherein the processor-executable routines, when executed by the processing system, cause the processing system to:
acquire a dynamic series of MRI images of a region of interest of a subject utilizing an MR scanner;
automatically identify one or more anatomical features in the region of interest in the dynamic series of MRI images; and
automatically refine the one or more anatomical features for use in generating a prescription for a subsequent scan of the region of interest with the MR scanner, wherein the refining of the one or more anatomical features is based on a consistency in position over time for the one or more anatomical features identified.

20. A non-transitory computer-readable medium, the computer-readable medium comprising processor-executable code that when executed by a processing system comprising one or more processors, causes the processing system to:
acquire a dynamic series of magnetic resonance imaging (MRI) images of a region of interest of a subject utilizing an MR scanner;
automatically identify one or more anatomical features in the region of interest in the dynamic series of MRI images; and
automatically refine the one or more anatomical features for use in generating a prescription for a subsequent scan of the region of interest with the MR scanner, wherein the refining of the one or more anatomical features is based on a consistency in position over time for the one or more anatomical features identified.
